(19) United States
(12) Patent Application Publication
(10) Pub. No.: US 2004/0053294 A1
(43) Pub. Date: Mar. 18, 2004

(54) Methods for identifying target genes, gene expression profiles, and biochemical/signal transduction pathways associated with specific patterns of hemodynamic shear stress and atherogenesis and uses thereof

(76) Inventors: Peter F. Davies, Haverford, PA (US); Denise C. Polacek, Wynnewood, PA (US)

Correspondence Address: Licata & Tyrrell P.C., 66 East Main Street, Marlton, NJ, 08053, US

(21) Appl. No.: 10/430,109

(22) Filed: May 5, 2003

Related U.S. Application Data
(60) Provisional application No. 60/377,925, filed on May 3, 2002.

(51) Int. Cl. C12Q 1/68

(52) U.S. Cl. 435/6

(57) ABSTRACT

Differentially expressed genes and profiles of differentially expressed genes associated with hemodynamic shear stress in vivo in mammalian vasculature are provided. Methods for identifying such profiles and using these profiles to identify and target regions of the vasculature with anti-atherogenic therapies are provided. Also provided are methods of identifying agents which modulate a gene of the profile for use in preventing atherogenesis and treating atherosclerosis.

### FIELD OF THE INVENTION

Differential gene expression profiles have now been identified in endothelial cells isolated from hemodynamically distinct regions of the mammalian aorta. The present invention provides methods for identifying genes and gene products, gene expression profiles, and biochemical/signal transduction pathways associated with specific hemodynamic shear stress areas and/or atherogenesis in the mammalian vasculature. The present invention also provides methods for identifying hemodynamically distinct regions of mammalian vasculature based upon the gene expression profile of endothelial cells in a selected region of the vasculature. Methods for identifying agents and using such agents to modulate the gene expression profile of endothelial cells under specific patterns of hemodynamic stress in the vasculature are also provided. It is expected that such agents will be useful in the prevention of atherogenesis.

### BACKGROUND OF THE INVENTION

Atherosclerosis is a disease of arteries that is responsible for most cardiovascular-related morbidity and mortality. While many atherosclerotic lesions are mild and cause little harm, those that progressively obstruct the passage of blood can reduce oxygen delivery to levels below the needs of the tissue (e.g., angina pain when coronary arteries are affected) or precipitate an acute ischemia (e.g., heart attack, stroke) when the obstruction forms on a destabilized lesion surface. Another catastrophic consequence of advanced lesions is the weakening of the artery wall, leading to pressure-induced ballooning (aneurysm) and potential rupture.

It is known that hemodynamics determines the location of atherogenic lesions. Local vessel geometry (e.g., arterial branching and curvatures), and constraint of vessel motion by surrounding tissues (e.g., coronary arteries) lead to flow instabilities and separations that correlate with sites of lesion development.

The one-cell thick layer at the interface between flowing blood and the artery wall is called the endothelium. Research has shown that the endothelium is not simply a passive barrier but rather is a multifunctional effector of systemic and vessel wall biology and is a very sensitive responder to the local environment. The endothelium is directly exposed to the hemodynamic shear stresses associated with all of the different flow characteristics found in the circulation.

Endothelial cell responses to the hemodynamic environment have been disclosed to be heterogeneous despite exposure of the cells to a substantially identical bulk flow field in vitro, or location of the cells in a predicted uniform hemodynamic environment in vivo. Prominent examples of the heterogeneous cell responses include the expression of VCAM-protein in vivo (Walpola et al., 1995, Arterioscler. Thromb. Vasc. Biol. 15:2-10) and in vitro (Ohtsuka et al., 1993, Biochem. Biophys. Res. Comm. 193:303-310), VCAM-1 mRNA expression in vivo (McKinsey et al., 1995, FASEB J. A343), ICAM-1 protein expression in vivo (Walpola et al., 1995, Arterioscler. Thromb. Vasc. Biol. 15:2-10) and in vitro (Nagel et al., 1994, J. Clin. Invest. 94:885-891), elevation of intracellular calcium ([Ca ²⁺]_{I}) measured in vitro (Geiger et al., 1992, Am. J. Physiol. 262:C1411-C1417; Shen et al., 1992, Am. J. Physiol. 262:C384-C390) and in vivo (Falcone et al., 1993, Am. J. Physiol. 264:H653-659), induction of synthesis and nuclear localization of c-fos in vitro (Ranjan et al., 1993, Biochem. Biophys. Res. Comm. 196:79-84), expression of major histocompatibility complex (MHC) antigens in vitro (Martin-Mondiere et al., 1989, ASAIO Trans. 35:288-290), inhibition of endothelial cell division in vitro (Ziegler et al., 1994, Arterioscler. Thromb. 14:636-643), and re-localization of the Golgi apparatus and microtubule organizing center (MTOC) in vitro (Coan et al., 1993, J. Cell Sci. 104:1145-1153). In each of these cases, high levels of response in one cell or a group of cells were accompanied by absent or diminished responses in adjacent cells of the same endothelial monolayer.

In vitro, nominal flow characteristics are defined by the geometry of the experimental system (e.g., flow tube, parallel plate, cone and plate, etc.). The average wall shear stress and shear stress gradient values can be accurately estimated or directly measured (Dewey et al., 1981, J. Biomech. Eng. 103:177-188; Davies et al., 1986, Proc. Nat. Acad. Sci. USA 83:2114-2118; Olesen et al., 1988, Nature 331:168-170; DePaola et al., 1992, Arterioscler. Thromb. 12:1254-1257). Although the flow characteristics are more complex in vivo, average shear stress values can be estimated from vessel geometry and flow rates (Zarins et al., 1983, Circ. Res. 53:502-514). Such measurements demonstrate that, although all of the cells in a given region of the monolayer are estimated to be subject to very similar shear stresses calculated from bulk flow characteristics, there are substantial cell-to-cell differences in acute and chronic responses to flow. If, as a significant number of experiments demonstrate, the responses are related to hemodynamic forces, it has not been determined what accounts for the heterogeneous responses.

In vitro flow chamber models of disturbed and undisturbed blood flow have recently been used to identify regionally defined differential expression of early response genes (DePaola et al., 1999, Proc. Natl. Acad. Sci. USA, 96:3154-3159). In regional differential gene expression studies during flow in vitro, endothelial cells are typically isolated by scraping the regions of interest. If enough cells are recovered, quantitative estimates of regional up- or down-regulation of gene expression (i.e., an average from all of the cells isolated from a particular location) can be made by northern blot analyses using specific nucleic acid probes for each gene of interest.

A useful alternative for analyzing the smaller numbers of cells typically present in defined hemodynamic regions is differential-display PCR (ddPCR; Liang et al., 1992, Science, 257:967-971), which uses reverse-transcription PCR (RT-PCR) to amplify all expressing genes in the cell population. This allows evaluation of differential expression of multiple genes when PCR products derived from cells in different hemodynamic regions are displayed together (e.g., as in Topper et al., 1996, Proc. Natl. Acad. Sci. USA, 93:10417-10422). Although ddPCR can be imprecise for quantitation of expression, this method has been used to identify differentially-expressed genes (e.g., Topper et al., 1997, Proc. Natl. Acad. Sci. USA, 94:9314-9319; Topper et al., 1997, J. Clin. Invest., 99:2942-2949; Topper et al., 1997, Proc. Natl. Acad. Sci. USA, 94, 9314-9319).

While such regional differential gene expression studies as described above are of value, the hemodynamic effects which modulate endothelial gene expression through spatial and temporal shear-stress relationships are ultimately defined locally at the surface of individual endothelial cells. Surface topographies, and consequently the magnitudes and gradients of shear-stresses, vary considerably from cell to cell (Barbee et al., 1995, Am. J. Physiol., 268:H1765-H1772). Atomic force microscopy (AFM) and computational fluid dynamics (CID) have been used to detail the geometry of living endothelial cell surfaces in vitro and in situ and to calculate the sub-cellular localized force distribution (Barbee et al., 1995, Am. J. Physiol. 268:H1765-H1772; Davies, 1995, Physiol. Rev. 75:519-560; Davies et al., 1997, Ann. Rev. Physiol. 59:527-549). Results of these studies indicated that microscopic hydrodynamic forces acting on individual endothelial cells vary considerably from cell to cell and within different regions of a single cell.

An important implication of these findings is that expression of a limited number of genes in even only a few endothelial cells can dominate vascular physiology and vascular pathogenesis. However, the identity of such genes has been complicated by “dilution” of mRNA transcribed from such genes by more numerous mRNA species, particularly when the pool of cells from which mRNA is isolated includes only a few “dominant” cells.

Accordingly, little is currently known about the genes involved in mediating the heterogeneous responses of endothelial cells to hemodynamic forces. Thus, there is an unmet need in the art for methods and compositions related to focal hydrodynamic stress-related regulation of gene expression which are useful in the development of methods for the prevention and treatment of atherosclerosis and other cardiovascular diseases in humans.

### SUMMARY OF THE INVENTION

An object of the present invention is to provide a method for identifying a gene, a gene product, a gene expression profile and/or a gene product biochemical/signal transduction pathway associated with a specific pattern of hemodynamic shear stress and/or predictive of susceptibility to atherogenesis in the mammalian vasculature. In this method of the present invention, vascular endothelial cells from a region of disturbed blood flow in the vasculature are isolated. These endothelial cells are referred to herein as disturbed-flow endothelial cells. Vascular endothelial cells from a region of relatively undisturbed blood flow in the vasculature are also isolated. These endothelial cells are referred to herein as undisturbed-flow endothelial cells. Gene expression profiles are then generated for both the disturbed-flow endothelial cells and the undisturbed-flow endothelial cells and these profiles are compared to identify genes differentially expressed in the disturbed-flow endothelial cells versus the undisturbed-flow endothelial cells and vice versa. Differential gene expression profiles of disturbed-flow endothelial cells versus undisturbed-flow endothelial cells serves as a means for identifying hemodynamically distinct regions of the vasculature. Genes, gene products and/or gene products of a biochemical/signal transduction pathways identified as being differentially expressed between the disturbed-flow endothelial cells and the undisturbed-flow endothelial cells also serve as potential targets for modulation of gene and/or gene product expression in specific hemodynamic regions of the vasculature and/or prevention of atherogenesis.

Accordingly, another object of the present invention is to provide a method for identifying hemodynamically disturbed regions of mammalian vasculature which comprises isolating endothelial cells from a selected region of the vasculature, generating a gene expression profile of the isolated endothelial cell or cells, and comparing this generated profile to a gene expression profile of disturbed-flow endothelial cells and undisturbed-flow endothelial cells, wherein a generated profile similar to the gene expression profile of disturbed-flow endothelial cells is indicative of the selected region being a region of hemodynamically disturbed flow and a generated profile similar to the gene expression profile of undisturbed-flow endothelial cells is indicative of the selected region being a region of hemodynamically undisturbed flow. This method is particularly useful in identifying regions of the vasculature to be targeted with anti-atherogenic agents.

Another object of the present invention is to provide methods for identifying agents which modulate a gene, gene product such as RNA or a protein, and/or a gene or gene product of a biochemical/signal transduction pathway that has been identified through analysis of the transcriptional profiles generated as described herein. In a preferred embodiment, agents identified will modulate expression of the gene, gene product and/or gene or gene product of a biochemical/signal transduction pathway in a manner which mimics gene expression in undisturbed flow endothelial cells. Such agents are expected to be useful in the prevention of atherogenesis and the treatment of atherosclerosis.

### DETAILED DESCRIPTION OF THE INVENTION

Atherosclerosis is a disease of arteries that is responsible for most cardiovascular-related morbidity and mortality. The focal origin of atherosclerosis and its localization to predictable locations in the arterial tree is strongly correlated with regions of complex hemodynamics that include flow separation/reattachment and flow instabilities. The spatial and temporal distribution of forces acting at the hemodynamic interface, the endothelium, can be measured over regions of many cells.

Endothelium from two regions of the adult pig aorta, from sites of disturbed flow and undisturbed flow, were compared for differential gene expression using cDNA microarrays. These microarrays permit measurement of many genes as a function of regional hemodynamics, also referred to herein as spatial genomics. Accordingly, differential transcription profiling of 15,000 genes was studied in endothelium isolated from the 2 hemodynamically-distinct regions of the pig aorta. It was found that less than 200 genes (<1.5%) were differentially expressed with the majority being suppressed in the lesion susceptible location. Suppression of these genes in indicative of an atheroprotective role in the proteins encoded thereby.

Advances in single cell and mRNA amplification techniques and methods of obtaining quantitative profiles of gene expression (e.g., transcriptional profiles) including the use of gene arrays for high throughput analyses now allows one to address endothelial heterogeneity in a very detailed (e.g., single cell and small groups of cells) yet comprehensive (multiple genes, high-throughput) approach. Accordingly, to refine regional profiling to fewer and fewer cells, linear amplification of mRNA obtained from single endothelial cells was examined.

Again endothelial cells lining the pig aorta were isolated from hemodynamically distinct regions, one with minimal hemodynamic stress and one with complex stress patterns. Amplified mRNA from these cells were labeled and used to screen gene arrays. Comparison of the gene expression profiles from cells in these two areas identified a number of genes that showed differential expression.

In particular, the following genes associated with cell cycle and death were identified as being down-regulated in atherosusceptible or disturbed flow arterial regions: TNF-beta, TNF-rec factor 6, Cdk inhibitor, PDGFb receptor, C-myc, IGF, IGF BP2, EGD precursor, EWS, Egr-1, Hevin, APRF DNA binding, SAT B1 (MAR/SAR DNA BP), NFX1 DNA BP and Suppressor of hairless. The following genes associated with cell cycle and death were identified as being up-regulated in atherosusceptible or disturbed flow arterial regions: FGF receptor 2 (K-sam), FGF activating protein, GCSF, helicase, Nip3, Ras and related GTP BP, endothelin-1, ET-1 receptor, KRAB Zn finger protein, HGF (Scatter Factor), cdc44, cdc46, adenosuccinylate synthetase, and uracil-DNA glycolase.

The following genes associated with signaling were identified as being down-regulated in atherosusceptible or disturbed flow arterial regions: CL100 protein tyrosine kinase, protein tyrosine phosphatase, PKC inhibitor, MEF 2B, mitochondrial ATP binding cassette 2, mitochondrial ATP synthase, and Ras-related GTP BP. The following genes associated with signaling were identified as being up-regulated in atherosusceptible or disturbed flow arterial regions: c-cbl, tyrosine phosphatase delta and slowpoke alpha.

The following genes associated with the extracellular matrix were identified as being down-regulated in atherosusceptible or disturbed flow arterial regions: alpha5-integrin, Hsp46, chondromodulin, and collagen type 2 alpha 1. The following genes associated with the extracellular matrix were identified as being up-regulated in atherosusceptible or disturbed flow arterial regions: TIMP4, collagen type 2 and collagen type VI.

The following genes associated with the cytoskeleton and trafficking were identified as being down-regulated in atherosusceptible or disturbed flow arterial regions: fibrillin, vimentin, vacuolar H+ ATPase, H+ ATP synthase subunit b, and Cd63. The following genes associated with the cytoskeleton and trafficking were identified as being up-regulated in atherosusceptible or disturbed flow arterial regions: LIM kinase, ELP-1 and UDP-glucose glycoprotein glucosyl transferase.

The following genes associated with transcription were identified as being down-regulated in atherosusceptible or disturbed flow arterial regions: NfkappaB, Hnup 153, and HXBP-1 transcription factor. The following genes associated with transcription were identified as being up-regulated in atherosusceptible or disturbed flow arterial regions: basic transcription factor BTF2p44, ILF transcription factor and Zpf-29.

The following genes associated with reduction and oxidation or Redox were identified as being down-regulated in atherosusceptible or disturbed flow arterial regions: eNOS, Cu/Zn SOD, NADP ubiquinone oxidoreductase and thioredoxin peroxidase. The following genes associated with reduction and oxidation or Redox were identified as being up-regulated in atherosusceptible or disturbed flow arterial regions: 15-S-lipoxygenase and cytochrome p450.

The following genes associated with immunological markers were identified as being down-regulated in atherosusceptible or disturbed flow arterial regions: MHC class 1 and class II, Hsp 89alpha, Hsp70, B2-microglobulin and TNFbeta. The immunological marker gene, cyclophilin A was identified as being up-regulated in atherosusceptible or disturbed flow arterial regions.

In addition, ABC2, a gene associated with lipid synthesis was identified as down-regulated in atherosusceptible or disturbed flow arterial regions. The following genes associated with lipid synthesis were identified as up-regulated in atherosusceptible or disturbed flow arterial regions: squalene synthase, IPP isomerase and L-3-hydroxacyl CoA dehydrogenase.

Accordingly, classification of the cellular functions performed by the products of these genes identified biochemical/signal transduction pathways that are believed to be affected by hemodynamic stress. For example, structural properties of the cells are believed to be affected, since differential expression was observed for genes whose products are involved in the cytoskeleton, trafficking and the extracellular matrix. The cell cycle is also believed to be altered in cells undergoing stress and cell death is expected to have a role in lesion formation. These changes are believed to be affected by differential signal transduction pathways since there is also differential expression for a number of signaling molecules. The surface and membranes of stressed cells are also believed to exhibit different characteristics since several cell surface markers, including ones with a role in the immune system, are differentially expressed. Genes whose products are involved in lipid biosynthesis also displayed a differential response. The effect of hemodynamic stress on the oxidation and reduction of molecules also appears to be manifested through the differential regulation of genes involved in redox function. Further, differential expression may be mediated through selective regulation by transcription factors.

Comparisons of hemodynamically distinct regions of the aortas of 8 animals were conducted by isolating endothelial RNA from each of the regions, amplifying the nucleotide linearly, and hybridizing to human cDNAs on the microarrays.

Data from this study were analyzed in two different databases involving different statistical analyses. The first was ArrayStat, a treatment that ignores statistical association between the two regions. The second was SAM, a treatment that is a paired analysis animal by animal. Because both analyses are powerful, both datasets were used to mine genes of differential expression.

Table 1 below outlines the total genes examined and the total number of genes within a functional category of genes identified as being differentially expressed by ArrayStat, SAM and both analyses.

TABLE I
Endothelial Genes Identified as Differentially Expressed by
Disturbed Flow (vs Undisturbed Flow) in the Pig Aorta
by Biological Classification

| Biological | | | | # Identified |
|---|---|---|---|---|
| Class or Key | # on | # Identified | # Identified | by ArrayStat |
| Word | Array | by ArrayStat | by SAM | & SAM |
| All Genes | 13824 | 1048 | 1881 | 780 |
| Adhesion | 298 | 24 | 38 | 18 |
| Apoptosis | 244 | 29 | 38 | 20 |
| Channel | 105 | 15 | 20 | 13 |
| Coagulation | 63 | 6 | 5 | 3 |
| (coag*) | | | | |
| Complement | 71 | 11 | 18 | 10 |
| Cytokine | 149 | 12 | 21 | 8 |
| Cytoskeleton | 489 | 38 | 73 | 32 |
| Endocytosis | 22 | 2 | 1 | 1 |
| Extracellular | 270 | 29 | 37 | 19 |
| Matrix | | | | |
| Focal Adhesion | 17 | 4 | 3 | 2 |
| Glutathione | 47 | 5 | 9 | 5 |
| Growth Factor | 238 | 28 | 44 | 23 |
| Immune Response | 136 | 17 | 26 | 15 |
| Inflammation | 150 | 14 | 18 | 10 |
| Interleukin | 90 | 9 | 10 | 4 |
| Ion Channel | 16 | 3 | 4 | 3 |
| Junction | 70 | 7 | 10 | 6 |
| Lipid | 145 | 12 | 17 | 10 |
| Lipoprotein | 35 | 4 | 7 | 4 |
| Macrophage | 26 | 2 | 0 | 0 |
| Motility | 116 | 13 | 22 | 10 |
| NFκB | 51 | 2 | 10 | 2 |
| Nitric Oxide | 14 | 1 | 3 | 1 |
| Oxidation | 166 | 13 | 23 | 9 |
| (oxid*) | | | | |
| Oxide | 50 | 7 | 13 | 7 |
| Peroxide | 34 | 6 | 10 | 6 |
| Proliferation | 391 | 39 | 60 | 26 |
| Prostaglandin | 36 | 1 | 6 | 1 |
| Signal | 2430 | 220 | 362 | 170 |
| Transduction | | | | |
| Superoxide | 18 | 3 | 5 | 3 |
| TNF | 69 | 5 | 7 | 4 |
| Transcription | 800 | 66 | 114 | 50 |
| Transcription | 557 | 47 | 83 | 36 |
| Factor | | | | |
| Transport | 531 | 56 | 75 | 43 |
| Vesicle | 129 | 6 | 9 | 5 |

Sufficient native RNA was also available to measure gene expression for a sample of genes believed to be differentially expressed in atherosusceptible or disturbed flow arterial regions by QRT-PCR. QRT-PCR analysis served as a validation of the array data. These data are depicted in Table 2.

TABLE 2
QRT-PCR Validation

| | | | | | | | | | | Ratio | p Value |
|---|---|---|---|---|---|---|---|---|---|---|---|
| | Pig | | | | | | | | P | in | in |
| Gene | 1 | Pig 2 | Pig 3 | Pig 4 | Pig 5 | Pig 6 | Mean | SD | Value | Array | Array |
| glutathione | 1.39 | 1.07 | 1.19 | 1.26 | 1.17 | 1.66 | 1.29 | 0.21 | 0.0196 | 11.82 | 0 |
| peroxidase | | | | | | | | | | | |
| GPX3 | | | | | | | | | | | |
| complement | 1.37 | 5.02 | 1.53 | 3.81 | 2.77 | 2.34 | 2.81 | 1.28 | 0.0096 | 5.2 | 0 |
| component C1r | | | | | | | | | | | |
| C1r | | | | | | | | | | | |
| brain-derived | 1.61 | 8.06 | 1.11 | 8.59 | 5.74 | 3.03 | 4.69 | 3.25 | 0.0387 | 1.88 | 2.36E−07 |
| neurotrophic | | | | | | | | | | | |
| factor | | | | | | | | | | | |
| BDNF | | | | | | | | | | | |
| complement | 0.18 | 0.18 | 0.15 | 0.58 | 0.25 | 0.34 | 0.28 | 0.16 | 0.0001 | 0.27 | 0 |
| component C3 | | | | | | | | | | | |
| early growth | 0.71 | 0.65 | 0.49 | 0.43 | 0.31 | 0.26 | 0.47 | 0.18 | 0.0008 | 0.49 | 1.97E−07 |
| response | | | | | | | | | | | |
| protein 1 | | | | | | | | | | | |
| (zinc finger | | | | | | | | | | | |
| pro) | | | | | | | | | | | |
| apolipoprotein | 0.14 | 0.1 | 0.18 | 0.56 | 0.27 | 0.23 | 0.25 | 0.17 | 0.0001 | 0.31 | 4.88E−15 |
| E | | | | | | | | | | | |
| APOE | | | | | | | | | | | |
| connexin 43 | 0.46 | 0.87 | 0.35 | 0.52 | 0.42 | 0.61 | 0.54 | 0.19 | 0.0017 | 0.66 | 0.00367 |
| cytochrome | 0.66 | 0.89 | 0.92 | 0.9 | 0.41 | 0.52 | 0.72 | 0.22 | 0.025 | 0.24 | 0.00124 |
| P450 | | | | | | | | | | | |
| monooxygenase | | | | | | | | | | | |
| CYP2J2 | | | | | | | | | | | |
| MHC class II | 0.65 | 0.49 | 0.59 | 0.35 | 0.41 | 0.52 | 0.5 | 0.11 | 0.0001 | 0.53 | 1.28E−05 |
| antigen | | | | | | | | | | | |
| HLA-DRB4 | | | | | | | | | | | |
| beta-2 | 0.67 | 0.23 | 1.22 | 0.4 | 0.23 | 0.49 | 0.54 | 0.37 | 0.0292 | 0.49 | 3.02E-10 |
| adrenergic | | | | | | | | | | | |
| receptor | | | | | | | | | | | |
| Prostacyclin | 0.78 | 0.6 | 0.52 | 1.22 | 0.44 | 0.37 | 0.66 | 0.31 | 0.0419 | 0.76 | 0.056 |
| Stimulating | | | | | | | | | | | |
| Factor? | | | | | | | | | | | |
| transforming | 0.64 | 0.81 | 0.61 | 0.78 | 0.62 | 0.44 | 0.65 | 0.13 | 0.0014 | 1.51 | 0.00246 |
| growth factor- | | | | | | | | | | | |
| beta 1, TGFB1 | | | | | | | | | | | |
| IGF-1 receptor | 1.54 | 2.17 | 1.39 | 1.44 | 1.34 | 1.16 | 1.51 | 0.35 | 0.0162 | 0.69 | 0.00149 |
| IGF1R | | | | | | | | | | | |
| von Willebrand | 1.52 | 12.45 | 43.96 | 2.72 | 2.04 | 12.29 | 11.58 | 16.38 | 0.174 | 5.92 | 0 |
| factor | | | | | | | | | | | |
| VWF | | | | | | | | | | | |
| HSP 90 | 3.4 | 1.09 | 1 | 2.19 | 1.61 | 1.52 | 1.8 | 0.89 | 0.0787 | 1.74 | 0.000112 |
| collagen VI | 0.5 | 1.56 | 0.71 | 0.62 | 0.63 | 0.73 | 0.79 | 0.39 | 0.2424 | 0.37 | 3.58E−10 |
| alpha-1 | | | | | | | | | | | |
| COL6A1 | | | | | | | | | | | |
| type I | 0.59 | 0.98 | 0.34 | 2.04 | 0.37 | 0.5 | 0.8 | 0.65 | 0.4908 | 0.32 | 3.13E−11 |
| collagen alpha | | | | | | | | | | | |
| 1 | | | | | | | | | | | |
| COL1A1 ?CBX6 | | | | | | | | | | | |
| profilin | 1.06 | 1.04 | 0.91 | 0.78 | 1.04 | 0.97 | 0.97 | 0.11 | 0.4809 | 2.57 | 5.74E−12 |
| PFN2 | | | | | | | | | | | |
| stem cell | 1.34 | 0.91 | 1.03 | 0.75 | 0.76 | 0.85 | 0.87 | 0.26 | 0.3422 | 1.42 | 0.00191 |
| factor MGF2 | | | | | | | | | | | |
| eNOS | 2.8 | 0.58 | 2.13 | 1.37 | 0.74 | 0.81 | 1.41 | 0.89 | 0.3151 | 0.69 | 0.00272 |
| transforming | 1.12 | 1.21 | 0.56 | 1.24 | 1.02 | 1.06 | 1.04 | 0.25 | 0.7432 | 2.10 | 4.54E−10 |
| growth factor | | | | | | | | | | | |
| beta 2, TGFB2 | | | | | | | | | | | |
| smooth muscle | 1.01 | 1.76 | 0.95 | 1.02 | 1.24 | 2.52 | 1.42 | 0.62 | 0.1593 | 1.69 | 0.000435 |
| protein 22- | | | | | | | | | | | |
| alpha | | | | | | | | | | | |
| low density | 4.18 | 0.22 | 3.83 | 0.76 | 0.48 | 0.41 | 1.65 | 1.84 | 0.4283 | 1.8 | 3.29E−05 |
| lipoprotein | | | | | | | | | | | |
| receptor, OLDLR | | | | | | | | | | | |
| 1 | | | | | | | | | | | |
| heme oxygenase | 3.12 | 0.41 | 0.29 | 3.81 | 2.83 | 1.34 | 1.97 | 1.49 | 0.1729 | 1.66 | 0.0014 |
| 1 | | | | | | | | | | | |
| plakoglobin | 1.25 | 1.03 | 1.07 | 0.79 | 0.95 | 1.34 | 1.07 | 0.20 | 0.4199 | 0.19 | 0 |
| glutathione S- | 0.72 | 0.71 | 0.33 | 1.13 | 1.33 | 1.34 | 0.93 | 0.41 | 0.6760 | 1.55 | 0.001457 |
| transferase | | | | | | | | | | | |
| NADH | 1.18 | 1.07 | 1.28 | 1.00 | 1.07 | 0.90 | 1.08 | 0.13 | 0.1864 | 1.40 | 0.0033 |
| ubiquinone | | | | | | | | | | | |
| oxidoreductase | | | | | | | | | | | |

Italicized text is indicative of up-regulation; emboldened text is indicative of down-regulation.

Seventy percent of the array samples were confirmed by QRT-PCR as depicted in Table 2, a percentage almost identical to that measured in an in vitro model validation study (Polacek et al. Physiological Genomics 2003 13:147-156).

Differential expression of multiple genes has also been confirmed at the protein level by immunocytochemistry.

Thus, as shown herein, differential expression of a gene, gene product profile of genes, and/or a gene or gene product of a biochemical/signal transduction pathway can be associated with hemodynamic shear stress in vivo and can be measured in mammalian vasculature. Accordingly, one aspect of the present invention relates to a method for identifying a gene, gene product, gene expression profile, and/or gene or gene product of a biochemical/signal transduction pathway associated with hemodynamic shear stress in vivo in mammalian vasculature. In this method endothelial cells are isolated from a region of disturbed blood flow in the vasculature. Vascular endothelial cells are also isolated from a region of undisturbed blood flow in the vasculature. Methods for isolating vascular endothelial cells are well known to those skilled in the art and can be performed routinely in accordance with such well known procedures. Gene expression profiles for the isolated disturbed flow endothelial cells and the isolated undisturbed flow endothelial cells are then generated. In a preferred embodiment, the profiles are generated on a single cell or small group of cells isolated from each region. The gene expression profiles of disturbed-flow endothelial cells and undisturbed-flow endothelial cells are then compared to identify a differentially expressed gene, gene product profile of differentially expressed genes, and/or a gene or gene product of a biochemical/signal transduction pathway in the disturbed-flow endothelial cells as compared to the undisturbed-flow endothelial cells. The differentially expressed gene, gene product profile of genes and/or a gene or gene product of a biochemical/signal transduction pathway is indicative of hemodynamic shear stress in the mammalian vasculature and predictive of susceptibility to atherosclerosis. By “gene product” it is meant to be inclusive of RNA, particularly mRNA, as well as proteins.

Another aspect of the present invention relates to a method for identifying a hemodynamically disturbed region of mammalian vasculature in vivo. In this method, endothelial cells are isolated from a selected region of the vasculature. A gene expression profile of the isolated endothelial cells is then generated and compared to gene expression profiles such as shown herein which have been generated for disturbed-flow endothelial cells and undisturbed-flow endothelial cells. Isolated endothelial cells exhibiting a gene expression profile similar to the gene expression profile of disturbed-flow endothelial cells is indicative of the selected region being a region of hemodynamically disturbed-flow while isolated endothelial cells exhibiting a gene expression profile similar to the gene expression profile of undisturbed-flow endothelial cells is indicative of the selected region being a region of hemodynamically undisturbed flow.

Regions of the vasculature identified as being hemodynamically disturbed serve as useful targets for anti-atherogenic therapies. Anti-atherogenic therapies may be targeted to these regions of the vasculature by various means. For example, in one embodiment, the agent may by linked to a peptide, protein or antibody to a cell surface marker differentially expressed in hemodynamically disturbed cells. Alternatively, a targeting implant such as a magnetic stent, rod or beads may be placed in the identified region to target the anti-atherogenic agent thereto. To identify a selected region of the vasculature for targeting with an anti-atherogenic agent, a gene expression profile of isolated endothelial cells from a selected region of the vasculature is generated. The generated gene expression profile is then compared to gene expression profiles such as taught herein for disturbed-flow endothelial cells and undisturbed-flow endothelial cells. Selected regions exhibiting a profile similar to the gene expression profile of disturbed-flow endothelial cells are preferred target regions for anti-atherogenic therapy.

The gene expression profiles of the present invention also provide means for identifying agents that modulate a gene, gene product, gene profile and/or gene or gene product of a biochemical/signal transduction pathway associated with an area of hemodynamic stress in the vasculature. Accordingly, another aspect of the present invention relates to screening assays for identifying agents that modulate a gene or genes, a gene products or products, and/or a gene or gene product of a biochemical/signal transduction pathway or pathways associated with hemodynamic stress in the vasculature. In these assays, gene expression profiles of disturbed-flow endothelial cells are generated in the absence and presence of a test agent. The generated gene expression profiles are then compared. Any change in the generated gene expression profile in the presence of the test agent is indicative of the agent modulating a gene or genes, a gene product or gene products and/or a gene or gene product of a biochemical/signal transduction pathway associated with an area of hemodynamic stress in the vasculature. Preferably, the presence of the agent induces a change in the gene expression profile of the disturbed flow endothelial cell which mimics gene expression in undisturbed flow endothelial cells. Such agents are expected to useful in preventing atherogenesis and/or treating atherosclerosis.

Identification of these differentially expressed gene profiles in vivo in hemodynamically stressed endothelial cells also provides useful information to identify, select and/or design new drugs for prevention of atherogenesis and/or treatment of atherosclerosis. Identification, selection and design of these new drugs will be based upon their ability to modulate one or more genes, gene products and/or gene or gene products of a biochemical/signal transduction pathway of the differentially expressed gene profiles as identified herein.

What is claimed is:

1. A method for identifying a gene, gene product expression profile, or a gene or gene product of a biochemical/signal transduction pathway associated with specific hemodynamic shear stress areas and predictive of susceptibility to atherosclerosis in vivo in mammalian vasculature comprising:
(a) isolating vascular endothelial cells from a region of disturbed blood flow in the vasculature;
(b) isolating vascular endothelial cells from a region of undisturbed blood flow in the vasculature;
(c) generating a gene expression profile for the disturbed-flow endothelial cells isolated in step (a);
(d) generating a gene expression profile for the undisturbed-flow endothelial cells isolated in step (b); and
(e) comparing the gene expression profile of disturbed-flow endothelial cells generated in step (c) with the gene expression, gene product, a profile of undisturbed flow endothelial cells generated in step (d) to identify a differentially expressed gene, gene product, a profile of differentially expressed genes, or a gene or gene product of a biochemical/signal transduction pathway in the disturbed-flow endothelial cells as compared to the undisturbed-flow endothelial cells indicative of a specific hemodynamic shear stress area in the mammalian vasculature.

2. A method for identifying a hemodynamically disturbed region of mammalian vasculature comprising:
(a) isolating endothelial cells from a selected region of the vasculature;
(b) generating a gene expression profile of the isolated endothelial cells; and
(c) comparing the gene expression profile generated in step (b) to gene expression profiles of disturbed flow endothelial cells and undisturbed flow endothelial cells, wherein a generated profile similar to the gene expression profile of disturbed flow endothelial cells is indicative of the selected region being a region of hemodynamically disturbed flow and a generated profile similar to the gene expression profile of undisturbed flow endothelial cells is indicative of the selected region being a region of hemodynamically undisturbed flow.

3. A method for identifying a selected region of the vasculature for targeting with an anti-atherogenic agent comprising:
(a) generating a gene expression profile of isolated endothelial cells from a selected region of the vasculature; and
(b) comparing the gene expression profile generated in step (a) to gene expression profiles of disturbed flow endothelial cells and undisturbed-flow endothelial cells, wherein a generated profile similar to the gene expression profile of disturbed-flow endothelial cells is indicative of the selected region being a target for anti-atherogenic therapy.

4. A method for identifying an agent which modulates a gene associated with hemodynamic stress in the vasculature comprising:
(a) generating gene expression profiles of disturbed-flow endothelial cells in the absence and presence of a test agent; and
(b) comparing the generated gene expression profiles wherein a change in the generated gene expression profiles in the presence of the test agent is indicative of the agent modulating a gene associated with hemodynamic stress in the vasculature.

5. The method of claim 4 wherein the change in the gene expression profile of the disturbed flow endothelial cells in the presence of the test agent mimics gene expression in undisturbed-flow endothelial cells.
